(19) United States
(12) Patent Application Publication
(10) Pub. No.: US 2021/0378539 A1
(43) Pub. Date: Dec. 9, 2021

(54) SYSTEM FOR RECOGNITION OF BIOLOGICAL ALTERATION IN HUMAN TISSUES

(71) Applicant: PAEGASUS MÉDICAL SA, Carouge (CH)

(72) Inventor: Stefano Nicolò CANEPA, Carouge (CH)

(21) Appl. No.: 17/287,518

(22) PCT Filed: Apr. 30, 2019
(86) PCT No.: PCT/EP2019/061088

§ 371 Date: Apr. 22, 2021

(30) Foreign Application Priority Data
Oct. 29, 2018 (EP) 18203207.8

(51) Int. Cl. A61B 5/0507; A61B 5/00

(52) U.S. Cl. CPC A61B 5/0507; A61B 5/4255; A61B 5/4227; A61B 5/4312; A61B 2562/0228; A61B 5/0028; A61B 5/0004; A61B 5/742; A61B 2560/0204; A61B 5/4381

(57) ABSTRACT

The present invention is directed to a system for recognition of biological alteration in human tissues using electromagnetic waves in the microwave range, the device comprising: a transmitter device (100) comprising at least one transmitting antenna (101), a transmitter (102), and a power supply (103); a receiving device (200) comprising at least one receiving antenna (201), a receiver (202), a pre-processing module (204), and a power supply (203); a microprocessor (301; 104) and a display (302; 105); wherein the transmitter device (100) and the receiving device (200) are configured to operate at a frequency comprised between 2.0 GHz and 3.0 GHz.

In a preferred embodiment, the operating frequency is comprised between 2.3 GHz and 2.5 GHz, and the device is suitable for the detection of a cancer in the human body, for example for the screening of prostate cancer, colorectal cancer, breast cancer, thyroid cancer.

The device according to the invention is capable of high sensitivity and accuracy of results and can detect not only the presence, but also the position of a cancer.

### FIELD OF THE INVENTION

The present invention is directed to a system for recognition of biological alteration in tissues, in particular it relates to a system making use of electromagnetic waves with an operating frequency comprised between 2.0 and 3.0 GHz.

### BACKGROUND OF THE INVENTION

The use of radio frequencies for recognition of biological alteration in tissues has been disclosed in several patent applications. WO 01/07909 (Vedruccio) discloses an apparatus comprising a coherent transceiver probe which generates radiofrequencies at three different wavelengths (436, 872 and 1308 MHz) and a spectrum analyzer. The probe is manually swept along the body of the patient and the decrease or disappearance of one of the three peaks is considered as an indicator of a type of alteration. Because of this, the three different wavelengths are considered essential. The apparatus, commercially known as Trimprob, was used by many doctors, but considered scarcely reliable since the results are strongly dependent on the operator.

EP 2 364 647 (Centro Studi e Ricerche Sant' Angela srl) discloses an apparatus for the detection of anomalies in biological tissues which tries to overcome the limits of the prior art. In particular, the apparatus addresses the problem of the lack of reliability by keeping the distance between transmitter and antenna constant. The apparatus of '647 tried also to overcome the dependency on the operator by moving the antenna automatically. The preferred frequency range is between 400 and 1000 MHz.

EP 2 465 428 (Medielma srl) is also addressed to the identical problem and tries to solve it by increasing the number of receiving antennas so that one of the receiving antennas is always connected to the transmitter and, consequently, the apparatus is capable of detecting the anomalies. The apparatus operates at a frequency lower than 700 MHz. In fact, the patent considers very important to irradiate the tissue in “near-filed conditions”. According to the patent, such condition can only be reached if the wavelength is lower than 700 MHz.

EP 3 257 439 (Medielma srl) discloses an evolution of the apparatus of '428, wherein the antenna radiating element is rotated of a predetermined angle both in clockwise and counter clockwise direction by means of a servo mechanism. Also in this case, the attempt is to reduce human activity and define a standard method for performing the analysis.

The results of the apparatuses according to the prior art, although useful, are considered as a preliminary screening, since the last word about the presence of a cancer is always left to other exams, e.g. NMR analysis colonoscopy, biopsy, etc. However, the above exams are expensive and invasive.

US 2017/007149 discloses an imaging system for detection of anomalies in the human body by the use of microwaves in the range from 10 to 26 GHz. The system is intended as a substitute of X-ray imaging and does not suggest the use of a microwaves in the frequency range from 2 to 3 GHz.

Thus, it would be highly desirable to be able to reduce the number of patients undergoing the above invasive exams. The present invention represents a very important step in this direction and provides an analytical tool with a reliability which is comparable to the more expensive imaging examinations.

The present invention is directed to a system for recognition of biological alteration in human tissues, more specifically for the detection of a cancer in some organs of the human body, without necessarily go for an imaging examination and it results in a diagnosis which is accurate, not only in terms of the correct assessment whether a cancer is present, but also about the location of the cancer.

### SUMMARY OF THE INVENTION

The present invention is directed to a system for recognition of biological alteration in human tissues, which system comprises a microwave transmitter, a microwave receiver, and a data processing unit and wherein the operating frequency of the microwave transmitter and receiver is comprised between 2.0 and 3.0 GHz. The use of a frequency within this range allows a very reliable diagnosis and localization of the cancer.

### BRIEF DESCRIPTION OF THE DRAWINGS

FIG. 1 is a block diagram of an embodiment of the device according to the invention.

FIG. 2 is a block diagram of another preferred embodiment of the invention.

FIG. 3 is a picture of a system according to the block diagram of FIG. 2.

FIG. 4 represents the screen of the transmitter of the system of FIG. 3.

### DETAILED DESCRIPTION OF THE INVENTION

The invention is directed to a system for the detection of tissue anomalies comprising: a transmitter device comprising at least one transmitting antenna, a transmitter, and a power supply; a receiving device comprising at least one receiving antenna, a receiver, a pre-processing module, and a power supply; and a data processing device comprising a microprocessor, a display and a power supply; wherein the transmitter device and the receiving device are configured to operate at a frequency comprised between 2.0 GHz and 3.0 GHz.

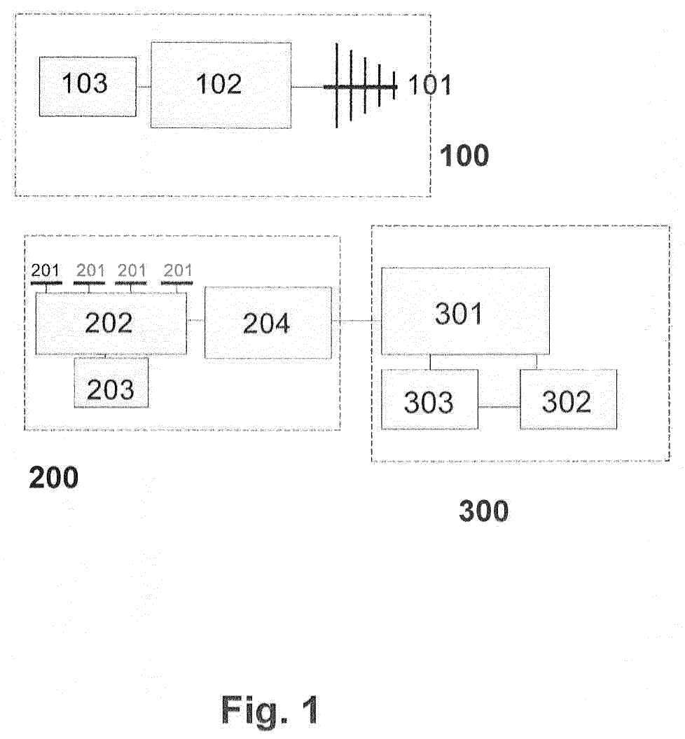

The block diagram of FIG. 1 shows a transmitter device 100 comprising an antenna 101, a transmitter 102, and a power supply 103; a receiver device 200, comprising four receiving antennas 201, a receiver 202, a pre-processing module 204, and a power supply 203; and a data processing device 300 comprising a microprocessor 301, a display 302 and a power supply 303.

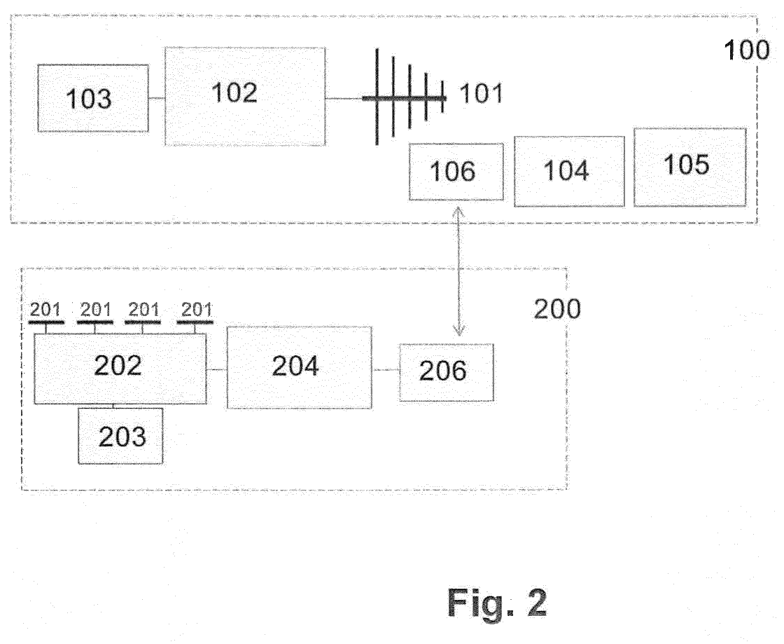

FIG. 2 represents another preferred embodiment of the invention wherein the data processing device 300 is integrated in the transmitter device 100 together with a Bluetooth transmitter/receiver 106. In this embodiment, the transmitter 100 comprises an antenna 101, a transmitter 102, a power supply 103, a Bluetooth transmitter/receiver 106, a microprocessor 104 and a display 105. The receiving device 200 also comprises a Bluetooth transmitter 206 and sends the data to microprocessor 104 through Bluetooth transmitter/receiver 106. In this embodiment, the operator can see directly on transmitter 100 the result of the analysis.

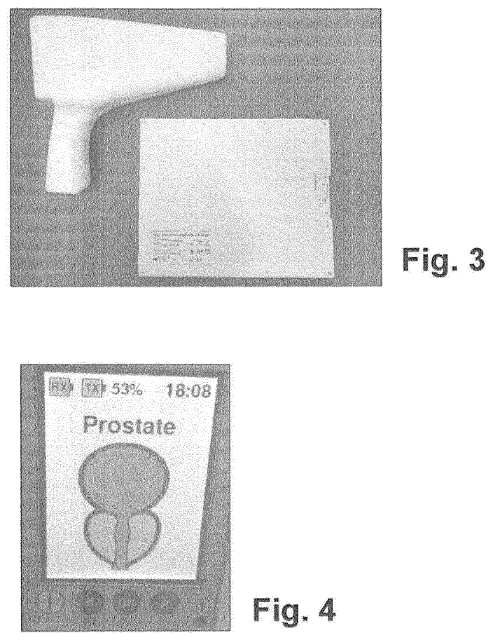

FIG. 3 is a picture of a prototype of the system according to the block diagram of FIG. 2. The transmitter device comprises a screen which receives data from the receiver device through a Bluetooth connection. This prototype is very light and can be easily transported in a bag. The receiver is of a size of about 0.25 m×0.20 m and contains four antennas positioned in a square configuration.

FIG. 4 shows the display of the transmitter of FIG. 3. Form the display the operator can follow the response of the analysis.

In one embodiment, the transmitter device comprises a single antenna, which is optionally a resonant cavity dipole antenna, or a directional antenna to be used in case of small organs, and a quartz oscillator to fix the specific emitted radio-frequency. Preferred types of directional antennas are Yagi antennas. Preferably, the directional antenna used in the transmitter of the present inventions irradiates with an angle comprised between 10° and 30°.

The transmitter is preferably structured to radiate a narrowband incident microwave frequency signal to irradiate a patient tissue. To the purpose of the present application, narrowband signal means a signal with a bandwidth Bw which is small enough to use the assumption that a response as a result of the interaction with the body can be considered constant within the bandwidth Bw, provided that 1/Bw is below the relaxation times of the irradiated patient biological tissues (typical Bw>1 KHz). The frequency radiated by the transmitter device is comprised in the range from 2.0 GHz to 3.0 GHz, for example from 2.2 GHz to 2.7 GHz, more preferably from 2.3 GHz to 2.5 GHz, most preferably around about 2.4 GHZ, which means from 2.35 GHz to 2.45 GHz.

The receiver device comprises at least one receiving antenna, a pre-processing module and a power supply.

The power supply preferably comprises rechargeable batteries. The receiving antenna(s) are tuned on the frequency of the transmitting antenna, i.e. from 2.0 GHz to 3.0 GHz, preferably from 2.2 GHz to 2.7 GHz, more preferably from 2.3 GHz to 2.5 GHz, most preferably around about 2.4 GHZ, which means from 2.35 GHz to 2.45 GHz. The receiving antennas can be either directional or omni directional; preferably, the receiving antennas are capable of covering 180°.

The pre-processing module preferably comprises a filter used to clean the signal excluding any other frequency different from the emitted frequency in order to eliminate electromagnetic noise of the environment. Signals from the system of receiving antennas are digitalized through the receiver and sent to the pre-processing module. From the pre-processing unit, the signal is sent to the processing unit which is preferably either in the transmitting device, or a separate device e.g. a laptop computer or an equivalent console.

The data processing unit through a processing algorithm provides parameters representing measures of the backscattered wave-field in term of different characteristics such as attenuation, polarization, resonances and interferences. Most preferably, the data processing unit is configured to run algorithms which determine the presence of nulls or minima (i.e. values below a threshold) in the received signal and associated with the presence of tissue anomalies, more specifically cancers. Examples of these algorithms are disclosed in EP 2 465 428, the entirety of which is incorporated herein by reference. The data processing unit can also comprise a display and a power supply, which preferably comprises rechargeable batteries. As an alternative, the receiving device comprises a Bluetooth transmitter and communicates the data to transmitting device which comprises a data processing unit and visualizes the results of the diagnosis on a screen.

The size of the receiver is compact. It is important to note that a receiver for the frequency of 2.4 GHz is small, since the size of an antenna is related to the wavelength λ of the radiation (2.4 GHz, λ=12 cm). If more than one receiving antenna is used, the length of the receiver is preferably less than 0.80 m, more preferably less than 0.50 m even more preferably less than 0.30 m.

Although multiple antennas can be present, it is also possible to have a single antenna in the receiving device. The use of a single antenna allows a further reduction of size of the receiver device. A receiver comprising a single receiving antenna has preferably a length which is less than 0.40 m, more preferably less than 0.30 m, even more preferably less than 0.25 m.

In any case, the width of the receiver is preferably less than 0.30 m and the thickness less than 0.030 m. This reduced size and thickness of the receiver allows it to be placed on the medical couch under the patient in the position corresponding to the organ to be examined.

In view of the reduced size of both the transmitter and the receiver, the device according to the invention can be easily transported to a patient bed or in a different location without any problem. In fact, the entire device can be placed in a bag, which allows easy transportation. The overall weight of the device according to the invention is very limited, e.g. lower than 10 kg, preferably lower than 5 kg, more preferably lower than 2 kg.

The device according to the invention can be used in any room and on any available medical couch and does not require a dedicated medical couch and room.

For this purpose, the device of the present invention is preferably provided with rechargeable batteries which allow operation of the device without the need of external power supply.

Another advantage of the device of the present invention is the capability of detecting the position of the cancer. In the case of prostate cancer, the device according to the invention is capable of determining whether the cancer is located on the left or on the right lobe. To obtain this information, to date it was necessary to perform an NMR analysis. Analogously, for colorectal cancer, the device according to the invention allows a diagnosis of the intestinal tract where the cancer is present.

The method of use of the device according to the invention is simple. The receiver is placed under the patient in correspondence to the organ to be checked. Then, the transmitter is turned on and placed in contact with the body as close as possible to the organ to be examined.

In a preferred embodiment, the screen of the system of FIGS. 3 and 4 will provide the operator with a screenshot requesting selection of the organ to be examined. A possible procedure for prostate and colon examination is herewith presented as a way of example. However, it is evident that a different presentation of information is possible. Similarly, other organs can be introduced in the menu of the system. If the organ is the prostate, the operator selects it and the screen will request examination of the left lobe of prostate. The operator will position the transmitter device in correspondence of the left lobe and will push a button on the screen to save the data. The screen will then request examination of the right lobe, and the operator will position the transmitter device on the right lobe and push the button again.

When selecting colon as the organ, the screen will request analysis of 6 different positions, which correspond to the relevant points to be examined: 1) right iliac fossa (caecum and ascending colon), 2) right hip (ascending colon and hepatic flexure), 3) mesogastrium (transverse colon, 4) left hip (splenic flexure and descending colon), 5) left iliac fossa (descending and sigmoid colon), 6) hypogastrium (sigmoid and colorectal colon). The operator will position the transmitter on each position in sequence and push the button to acquire the data.

Thus, in a preferred embodiment of the invention, the system comprises a software adapted to the examination of at least one of the following organs: prostate, colon, breast and thyroid. Preferably, the display of the system guides the examination and the operator by pushing a button saves the data measured by the system and indicating whether the examination is positive or negative. When all the area to be examined are completed the system provides an outcome of the examination (positive or negative) and possibly the area where the cancer is located (left or right lobe for prostate, the area of the colon for colon, etc.).

Preferably, the processor is adapted to store data of a number of examinations corresponding to at least one working day. For example, the processor can store at least 100 results of examinations. In this manner, the operator can perform several examinations and at the end of the day connect the system to a processor such as a computer and download the data. Thus, the system of the invention is preferably adapted to transfer the stored data to an external processor. Transfer is preferably performed by the Bluetooth connection (106, 206).

### Experimental Part

### Prostate Examination

155 urologic patients were evaluated by multiparametric NMR, and by the system of FIGS. 3 and 4 according to the invention (2.4 GHz). The results are confronted with the results of a prostate transrectal eco guided biopsy, which is considered the gold standard in prostate cancer diagnosis.

For each methodology, two different analyses were performed: a first analysis on the entire prostate (Table 1), and a second analysis to assess the laterality of a possible prostate cancer (Table 2).

TABLE 1

| | 2.4 GHz | NMR |
|---|---|---|
| True Positive (TP) | 19 | 15 |
| False Positive (FN) | 7 | 6 |
| True Negative (TN) | 129 | 130 |
| False Negative (FN) | 0 | 4 |

TABLE 2

| | 2.4 GHz | NMR |
|---|---|---|
| True Positive (TP) | 22 | 19 |
| False Positive (FN) | 10 | 8 |
| True Negative (TN) | 275 | 277 |
| False Negative (FN) | 3 | 6 |

The results on table 1 show that the device according to the invention did not produce any false negative and a number of false positive comparable to the number obtained by NMR. It is important to note that false negatives are the most dangerous cases, since when a false negative occurs, a person which is diagnosed sane, is in fact affected by a cancer. Tables 3 and 4 report a statistical evaluation of the data of table 1 and 2 respectively by calculating sensitivity, specificity, negative predictive value (NPV), positive predictive value (PPV), and accuracy, wherein:

Sensitivity=TP/(TP+FN)·100

Specificity=TN/(TN+FP)·100

PPV=TP/(TP+FP)·100

NPV=TN/(TN+FN)·100

Accuracy=(TP+TN)/(TP+FP+TN+FN)·100

TABLE 3
Statistical analysis of the results of Table 1

| | Sensitivity | Specificity | PPV | NPV | Accuracy |
|---|---|---|---|---|---|
| 2.4 GHz | 100 | 94.85 | 73.08 | 100 | 95.48 |
| NMR | 78.95 | 95.59 | 71.43 | 97.01 | 93.55 |

TABLE 4
Statistical analysis of the results of Table 2

| | Sensitivity | Specificity | PPV | NPV | Accuracy |
|---|---|---|---|---|---|
| 2.4 GHz | 88.00 | 96.49 | 68.75 | 98.92 | 95.81 |
| NMR | 76.00 | 97.19 | 70.37 | 97.88 | 95.45 |

From the data of tables 3 and 4 it is evident that the device of the present invention is highly reliable and allows a screening of patients to detect the presence of a prostate cancer in an easy and economical manner. The device is also suitable for recognition of alteration of other organs, for example for the screening of colorectal cancer, breast cancer, thyroid cancer.

### Colon Examination

It was performed a prospective single-center blinded study of consecutive adults undergoing colonoscopy. Before patients underwent colonoscopy, they were examined by the system according to the invention.

During the procedure the subjects is dressed. A hand-held device was moved over the abdomen and electromagnetic response of tissues signals were recorded (2.4 GHz). A single investigator performed the test using the system. Abnormal signals were identified and recorded as malignant or benign (adenoma, hyperplastic polyps or diverticula). Findings were compared with those from colonoscopy. Statistical analysis was then performed.

### Results

A total of 107 consecutive patients fulfilling the inclusion criteria were enrolled over a period of 5 months. The most frequent indication for colonoscopy was constipation, diarrhea, abdominal pain or fecal blood. The system according to the invention detected and characterized all 32 adenocarcinomas and polyps.

The method identified cancers and polyps with 96.97% sensitivity, 78.38% specificity, and 84.11% diagnostic accuracy, compared to colonoscopy. The positive predictive value was 66.67% and the negative predictive value 98.31%. Among the 107 subjects, there were 16 false positive results (14.95%) and 1 false negative (0.93%) result. The results are summarized in Table 5.

TABLE 5

| | Sensitivity | Specificity | PPV | NPV | Accuracy |
|---|---|---|---|---|---|
| 2.4 GHz | 96.97 | 78.38 | 66.67 | 98.31 | 84.11 |

Colonoscopy is a very invasive examination which requires a long preparation, while examination by the system according to the invention is easy to perform and does not require any preparation.

1. A system for the detection of tissue anomalies comprising:
a. a transmitter device comprising at least one transmitting antenna, a transmitter, and a power supply;
b. a receiving device comprising at least one receiving antenna, a receiver, a pre-processing module, and a power supply (203); and
c. a microprocessor and a display;
wherein the transmitter device and the receiving device are configured to operate at a frequency comprised between 2.0 GHz and 3.0 GHz.

2. The system according to claim 1 wherein the microprocessor and the display are integrated in the transmitter device together with a Bluetooth transmitter/receiver, and wherein the receiving device also comprises a Bluetooth transmitter/receiver.

3. The system according to claim 1, wherein the operating frequency is comprised between 2.1 GHz and 2.7 GHz.

4. The system according to claim 1, wherein the operating frequency is comprised between 2.3 GHz and 2.5 GHz.

5. The system according to claim 1, wherein the transmitter is structured to radiate a narrowband incident radiofrequency signal.

6. The system according to claim 1, wherein the transmitter device comprises a single antenna, and a quartz oscillator to fix the specific emitted radio-frequency.

7. The system according to claim 1, wherein the transmitting antenna is a directional antenna which irradiates with an angle comprised between 10° and 30°.

8. The system according to claim 1, wherein the power supply comprises a rechargeable battery.

9. The system according to claim 1, wherein the processor comprises a software adapted to the examination of at least one of the following organs: prostate, colon, breast and thyroid.

10. The system according to claim 1, wherein the microprocessor is adapted to store the results of a number of examinations.

11. The system of claim 10, wherein the microprocessor is adapted to store at least 100 results of examinations.

12. The system of claim 10, wherein the system is adapted to transfer the stored data to an external processor.
